(19) United States
(12) Patent Application Publication
(10) Pub. No.: US 2020/0163562 A1
(43) Pub. Date: May 28, 2020

(54) LOW NOISE FRONT-END FOR A HEART RATE MONITOR USING PHOTO-PLETHYSMOGRAPHY

(71) Applicant: Intel Corporation, Santa Clara, CA (US)

(72) Inventor: Philip Neaves, Wiltshire, Swindon (GB)

(73) Assignee: Intel Corporation, Santa Clara, CA (US)

(21) Appl. No.: 16/636,897

(22) PCT Filed: Aug. 13, 2018
(86) PCT No.: PCT/US2018/045849

§ 371 Date: Feb. 5, 2020

Related U.S. Application Data
(60) Provisional application No. 62/556,260, filed on Sep. 8, 2017.

(51) Int. Cl. A61B 5/024; H03F 3/45

(52) U.S. Cl. CPC A61B 5/02427; H03F 3/45071; H03F 2203/45022; H03F 2200/498; H03F 2203/45528; H03F 2203/45116; H03F 2203/45151; H03F 2200/129

(57) ABSTRACT

An apparatus is provided for monitoring heart rate. The apparatus comprises various components to effectively reduce photodiode capacitance. The apparatus includes: an amplifier; a first current source; a first pair of resistors coupled to the first current source and the amplifier; a pair of devices coupled to the first pair of resistors; a photo-diode coupled to the pair of devices; a second pair of resistors coupled to the pair of devices and the photo-diode; and a second current source coupled to the second pair of resistors.

### CLAIM OF PRIORITY

This application claims benefit of priority of U.S. Provisional Application No. 62/556,260 filed Sep. 8, 2017, titled “Low NOISE FRONT-END FOR A HEART RATE MONITOR USING PHOTO-PLETHYSMOGRAPHY,” and is incorporated by reference in its entirety.

### BACKGROUND

Photoplethysmography (PPG) based heart rate detection works by detecting reflected light from blood vessels as the blood vessels dilate and contract in sympathy with changing blood pressure associated with the heartbeat. The light is generated by a pulsed Light Emitting Diode (LED) which is placed against the skin (often a wrist) and detected by a photodiode also placed against the skin in near vicinity to the LED. Since the LED has a wide transmission angle and the emitted light is subject to scattering within the body, light reflects to the photodiode from extraneous sources such as bones as well as from the blood vessels. The signal component obtained from the light reflected from extraneous sources is commonly referred to as the DC component of the received signal. The undesired DC reflected component received is significantly greater than the signal from the blood vessel (e.g., the DC reflected component may be over 80 dB greater than the signal of interest which may typically be just 400 pA). The undesired DC component presents a number of issues. For example, amplifying the input signal to provide sufficient gain to the desired signal to detect it may lead to saturation in the amplifier stages of the PPG device. PPG devices may wobble during use. As such, variable motion artifacts are introduced into the received photo-currents making tracking of the desired signal from the undesired signal difficult.

### BRIEF DESCRIPTION OF THE DRAWINGS

The embodiments of the disclosure will be understood more fully from the detailed description given below and from the accompanying drawings of various embodiments of the disclosure, which, however, should not be taken to limit the disclosure to the specific embodiments, but are for explanation and understanding only.

FIG. 1 illustrates an ensemble of wearable devices including a Photoplethysmography (PPG) device with a low noise front-end, according to some embodiments of the disclosure.

FIG. 2 illustrates a conventional Transimpedance Amplifier (TIA) with photodiode.

FIG. 3 illustrates an apparatus with a common-gate stage to effectively reduce photodiode capacitance, in accordance with some embodiments.

FIG. 4 illustrates an apparatus with a fully-differential common-gate circuitry to effectively reduce photodiode capacitance, in accordance with some embodiments.

FIG. 5 illustrates an apparatus with current splitting resistors based fully-differential common-gate circuitry to effectively reduce photodiode capacitance, in accordance with some embodiments.

FIG. 6 illustrates a SoC (System-on-Chip) with a PPG device with common-gate stage to effectively reduce photodiode capacitance, according to some embodiments of the disclosure.

FIG. 7 illustrates a smart device or a computer system or a SoC (System-on-Chip) having common-gate circuitry to effectively reduce photodiode capacitance, according to some embodiments of the disclosure.

### DETAILED DESCRIPTION

Market potential in the emerging wearable wellness and sports monitoring space is vast. Accurate integrated heartrate and blood oxygen measurement circuitry is a vital part of increasing market share. A significant challenge for the present and the future is the fast and robust acquisition of cardiac waveforms from photodiodes using photoplethysmography or photoplethysmogram (PPG). PPG is an optically obtained volumetric measurement of an organ. Using traditional PPG measurement technology to wearable devices is non-optimal because they consume high power and circuit area. Additionally, wrist based wearable devices may introduce sharp motions due to user's wrist motion.

Various embodiments describe an apparatus which comprises a PPG device with a low noise front-end. In some embodiments, the low noise front-end comprises one or more transimpedance amplifiers (TIAs) that are used in the field of photoplethysmography for wearable optical heart rate monitors. The 1/f (one over frequency) noise performance of a signal chain (e.g., signal representing the heart rate) and the TIA play an important role in determining the accuracy and reliability of the measured heart-rate. The wanted signal, for example “heart rate” and the 1/f noise are generally in the same pass-band.

In some embodiments, in a PPG application, a light source (e.g., a light emitting diode (LED)) shines light into a user's skin under a wearable device (e.g., a smartwatch). In some embodiments, this light is shone at low duty cycles to save power (e.g., a short duration pulse to minimise power dissipation). Duty cycle is the ratio of on to off events (e.g., ratio of logic 1 to logic 0 etc.). The repetition rate of the pulse may be a few tens of Hertz (Hz) to up to a few kHz (Kilo Hz), in accordance with some embodiments. A person skilled in the art would appreciate that the higher the frequency (e.g., the repetition rate of the pulse) the better the resolution but the more the current consumed.

In some embodiments, the PPG device comprises a current-source that activates by light (e.g., photodiode) which generates an output current in response to the received light. In some embodiments, the PPG device comprises a common-gate circuitry (also referred to as the common-gate stage). In some embodiments, a TIA is described that uses a common-gate architecture that has low noise performance. A common-gate architecture allows the use of photodiodes with large capacitance and a TIA with large transimpedance gain. In some embodiments, the noise from the common-gate stage is made “common-mode” by two architectural choices: (i) a novel biasing technique using resistive current-splitting; and (ii) a fully differential TIA. In various embodiments, the resulting common-mode noise is no longer seen by a fully differential analog-to-digital converter (ADC) at the output of the TIA. As such, common-gate circuitry can be used in low noise TIA design. Note, conventional design wisdom is that using common-gate stage in a TIA design is a poor design choice and not realizable for low noise TIA design.

In the following description, numerous details are discussed to provide a more thorough explanation of embodiments of the present disclosure. It will be apparent, however, to one skilled in the art, that embodiments of the present disclosure may be practiced without these specific details. In other instances, well-known structures and devices are shown in block diagram form, rather than in detail, in order to avoid obscuring embodiments of the present disclosure.

Note that in the corresponding drawings of the embodiments, signals are represented with lines. Some lines may be thicker, to indicate more constituent signal paths, and/or have arrows at one or more ends, to indicate primary information flow direction. Such indications are not intended to be limiting. Rather, the lines are used in connection with one or more exemplary embodiments to facilitate easier understanding of a circuit or a logical unit. Any represented signal, as dictated by design needs or preferences, may actually comprise one or more signals that may travel in either direction and may be implemented with any suitable type of signal scheme.

Throughout the specification, and in the claims, the term “connected” means a direct connection, such as electrical, mechanical, or magnetic connection between the things that are connected, without any intermediary devices.

The term “coupled” means a direct or indirect connection, such as a direct electrical, mechanical, or magnetic connection between the things that are connected or an indirect connection, through one or more passive or active intermediary devices. The term “circuit” or “module” may refer to one or more passive and/or active components that are arranged to cooperate with one another to provide a desired function.

The term “signal” may refer to at least one current signal, voltage signal, magnetic signal, or data/clock signal. The meaning of “a,” “an,” and “the” include plural references. The meaning of “in” includes “in” and “on.”

The term “scaling” generally refers to converting a design (schematic and layout) from one process technology to another process technology and subsequently being reduced in layout area. The term “scaling” generally also refers to downsizing layout and devices within the same technology node. The term “scaling” may also refer to adjusting (e.g., slowing down or speeding up—i.e. scaling down, or scaling up respectively) of a signal frequency relative to another parameter, for example, power supply level.

The terms “substantially,” “close,” “approximately,” “near,” and “about,” generally refer to being within +/−10% of a target value. For example, unless otherwise specified in the explicit context of their use, the terms “substantially equal,” “about equal” and “approximately equal” mean that there is no more than incidental variation between among things so described. In the art, such variation is typically no more than +/−10% of a predetermined target value.

The term “device” may generally refer to an apparatus according to the context of the usage of that term. For example, a device may refer to a stack of layers or structures, a single structure or layer, a connection of various structures having active and/or passive elements, etc. Generally, a device is a three-dimensional structure with a plane along the x-y direction and a height along the z direction of an x-y-z Cartesian coordinate system. The plane of the device may also be the plane of an apparatus which comprises the device.

The term “adjacent” here generally refers to a position of a thing being next to (e.g., immediately next to or close to with one or more things between them) or adjoining another thing (e.g., abutting it).

Unless otherwise specified the use of the ordinal adjectives “first,” “second,” and “third,” etc., to describe a common object, merely indicate that different instances of like objects are being referred to, and are not intended to imply that the objects so described must be in a given sequence, either temporally, spatially, in ranking or in any other manner.

For the purposes of the present disclosure, phrases “A and/or B” and “A or B” mean (A), (B), or (A and B). For the purposes of the present disclosure, the phrase “A, B, and/or C” means (A), (B), (C), (A and B), (A and C), (B and C), or (A, B and C).

The terms “left,” “right,” “front,” “back,” “top,” “bottom,” “over,” “under,” and the like in the description and in the claims, if any, are used for descriptive purposes and not necessarily for describing permanent relative positions. For example, the terms “over,” “under,” “front side,” “back side,” “top,” “bottom,” “over,” “under,” and “on” as used herein refer to a relative position of one component, structure, or material with respect to other referenced components, structures or materials within a device, where such physical relationships are noteworthy. These terms are employed herein for descriptive purposes only and predominantly within the context of a device z-axis and therefore may be relative to an orientation of a device. Hence, a first material “over” a second material in the context of a figure provided herein may also be “under” the second material if the device is oriented upside-down relative to the context of the figure provided. In the context of materials, one material disposed over or under another may be directly in contact or may have one or more intervening materials. Moreover, one material disposed between two materials may be directly in contact with the two layers or may have one or more intervening layers. In contrast, a first material “on” a second material is in direct contact with that second material. Similar distinctions are to be made in the context of component assemblies.

The term “between” may be employed in the context of the z-axis, x-axis or y-axis of a device. A material that is between two other materials may be in contact with one or both of those materials, or it may be separated from both of the other two materials by one or more intervening materials. A material “between” two other materials may therefore be in contact with either of the other two materials, or it may be coupled to the other two materials through an intervening material. A device that is between two other devices may be directly connected to one or both of those devices, or it may be separated from both of the other two devices by one or more intervening devices.

Here, multiple non-silicon semiconductor material layers may be stacked within a single fin structure. The multiple non-silicon semiconductor material layers may include one or more “P-type” layers that are suitable (e.g., offer higher hole mobility than silicon) for P-type transistors. The multiple non-silicon semiconductor material layers may further include one or more “N-type” layers that are suitable (e.g., offer higher electron mobility than silicon) for N-type transistors. The multiple non-silicon semiconductor material layers may further include one or more intervening layers separating the N-type from the P-type layers. The intervening layers may be at least partially sacrificial, for example to allow one or more of a gate, source, or drain to wrap completely around a channel region of one or more of the N-type and P-type transistors. The multiple non-silicon semiconductor material layers may be fabricated, at least in part, with self-aligned techniques such that a stacked CMOS device may include both a high-mobility N-type and P-type transistor with a footprint of a single finFET.

For purposes of the embodiments, the transistors in various circuits and logic blocks described here are metal oxide semiconductor (MOS) transistors or their derivatives, where the MOS transistors include drain, source, gate, and bulk terminals. The transistors and/or the MOS transistor derivatives also include Tri-Gate and FinFET transistors, Gate All Around Cylindrical Transistors, Tunneling FET (TFET), Square Wire, or Rectangular Ribbon Transistors, ferroelectric FET (FeFETs), or other devices implementing transistor functionality like carbon nanotubes or spintronic devices. MOSFET symmetrical source and drain terminals i.e., are identical terminals and are interchangeably used here. A TFET device, on the other hand, has asymmetric Source and Drain terminals. Those skilled in the art will appreciate that other transistors, for example, Bi-polar junction transistors (BJT PNP/NPN), BiCMOS, CMOS, etc., may be used without departing from the scope of the disclosure.

It is pointed out that those elements of the figures having the same reference numbers (or names) as the elements of any other figure can operate or function in any manner similar to that described, but are not limited to such.

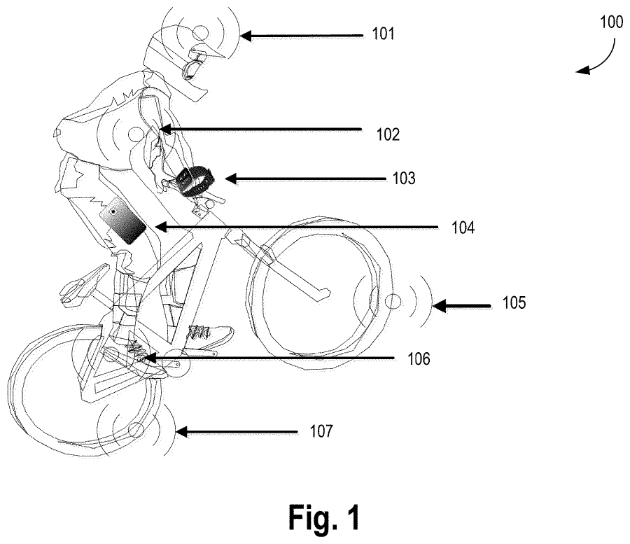

FIG. 1 illustrates an ensemble of wearable devices including a Photo-plethysmography (PPG) device with low noise front-end, according to some embodiments of the disclosure. In this example, ensemble 100 is on a person and his/her ride (here, a bicycle). However, the embodiments are not limited to such. Other scenarios of wearable devices and their usage may work with the various embodiments.

For example, a PPG device with apparatus to effectively reduce photodiode capacitance can be embedded into some other products (e.g., medical devices, ambulances, patient uniform, doctor's uniform, etc.) and can be controlled using a controller or a terminal device. The PPG device with apparatus to effectively reduce photodiode capacitance of some embodiments can also be part of a wearable device. The term “wearable device” (or wearable computing device) generally refers to a device coupled to a person. For example, devices (such as sensors, cameras, speakers, microphones (mic), smartphones, smart watches, medical devices, etc.) which are directly attached on a person or on the person's clothing are within the scope of wearable devices.

In some examples, wearable computing devices may be powered by a main power supply such as an AC/DC power outlet. In some examples, wearable computing devices may be powered by a battery. In some examples, wearable computing devices may be powered by a specialized external source based on Near Field Communication (NFC). The specialized external source may provide an electromagnetic field that may be harvested by circuitry at the wearable computing device. Another way to power the wearable computing device is electromagnetic field associated with wireless communication, for example, WLAN (Wireless Local Area Network) transmissions. WLAN transmissions use far field radio communications that have a far greater range to power a wearable computing device than NFC transmission. WLAN transmissions are commonly used for wireless communications with most types of terminal computing devices.

For example, the WLAN transmissions may be used in accordance with one or more WLAN standards based on Carrier Sense Multiple Access with Collision Detection (CSMA/CD) such as those promulgated by the Institute of Electrical Engineers (IEEE). These WLAN standards may be based on CSMA/CD wireless technologies such as Wi-Fi™ and may include Ethernet wireless standards (including progenies and variants) associated with the IEEE 802.11-2012 Standard for Information technology—Telecommunications and information exchange between systems—Local and metropolitan area networks—Specific requirements Part 11: WLAN Media Access Controller (MAC) and Physical Layer (PHY) Specifications, published March 2012, and/or later versions of this standard (“IEEE 802.11”).

Continuing with the example of FIG. 1, ensemble 100 of wearable devices includes device 101 (e.g., camera, microphone, etc.) on a helmet, device 102 (e.g., PPG device with apparatus to track and cancel DC offset, where the PPG device can be a pulse sensor, heartbeat sensor, blood oxygen level sensor, blood pressure sensor, or any other sensor such as those used in the fields of ECG (electrocardiography), EMG (electromyography), EOG (electrooculography, which is a detection of current associated with movement of the eyeball), ENG (electronystagmography), etc.) on the person's arm, device 103 (e.g., a smart watch that can function as a terminal device, controller, or a device to be controlled), device 104 (e.g., a smart phone and/or tablet in a pocket of the person's clothing), device 105 (e.g., pressure sensor to sense or measure pressure of a tire, or gas sensor to sense nitrogen air leaking from the tire), device 106 (e.g., an accelerometer to measure peddling speed), device 107 (e.g., another pressure sensor for the other tire). In some embodiments, ensemble 100 of wearable devices has the capability to communicate by wireless energy harvesting mechanisms or other types of wireless transmission mechanisms.

In some embodiments, device 102 comprises PPG device with a low noise front-end. In some embodiments, in a PPG application, a light source (e.g., a LED) shines light into a user's skin under a wearable device (e.g., a smartwatch). In some embodiments, this light is shone at low duty cycles to save power (e.g., a short duration pulse to minimise power dissipation). Duty cycle is the ratio of on to off events (e.g., ratio of logic 1 to logic 0 etc.). The repetition rate of the pulse may be a few tens of Hertz to up to a few kHz, in accordance with some embodiments. A person skilled in the art would appreciate that the higher the frequency (e.g., the repetition rate of the pulse) the better the resolution but the more the current consumed.

In some embodiments, the PPG device comprises a current-source circuitry that activates by light (e.g., photodiode) which generates an output current in response to the received light. In some embodiments, the PPG device comprises a common-gate circuitry (stage). In some embodiments, the noise from the common-gate stage is made “common-mode” by two architectural choices: (i) a novel biasing technique using resistive current-splitting; and (ii) a fully differential TIA. In various embodiments, the resulting common-mode noise is no longer seen by a fully differential ADC at the output of the TIA. As such, common-gate stage can be used in low noise TIA design. Note, conventional design wisdom is that using common-gate stage in a TIA design is a poor design choice and not realizable for low noise TIA design.

In some embodiments, the PPG device includes an antenna to transmit the processed data (e.g., the digitized data in modulated form from the output of the TIA) to a controller or a terminal device (e.g., a smart phone, laptop, cloud, etc.) for further processing. In some embodiments, the antenna may comprise one or more directional or omnidirectional antennas, including monopole antennas, dipole antennas, loop antennas, patch antennas, microstrip antennas, coplanar wave antennas, or other types of antennas suitable for transmission of Radio Frequency (RF) signals. In some multiple-input multiple-output (MIMO) embodiments, the antennas are separated to take advantage of spatial diversity.

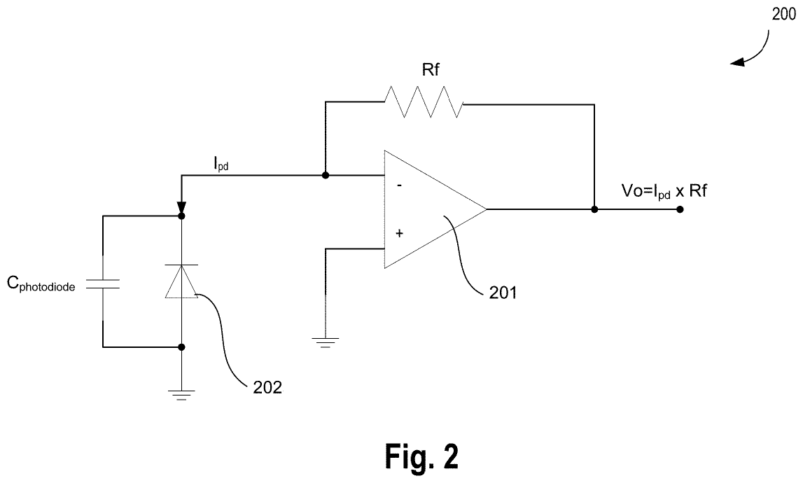

FIG. 2 illustrates a conventional Transimpedance Amplifier (TIA) 200 with a photodiode. TIA 200 includes an amplifier 201, feedback resistor Rf, and photodiode 202 coupled together as shown. Here, C_{photodiode} (Cpd) is the capacitance associated with the photodiode 202. The photodiode current Ipd results in an output voltage of Vo=Ipd×Rf. The conventional TIA suffers from the problem that large values of Rf (e.g., “gain”) and large photodiode capacitance Cpd cause the TIA to become unstable.

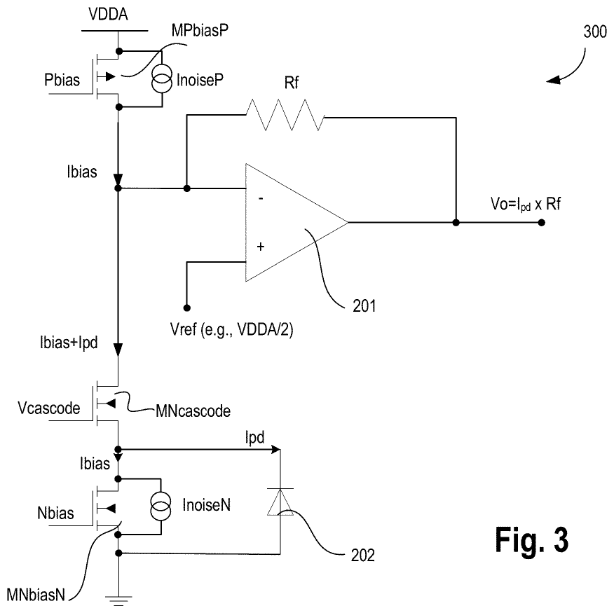

FIG. 3 illustrates apparatus 300 with common-gate stage to effectively reduce photodiode capacitance, in accordance with some embodiments. In some embodiments, apparatus 300 comprises amplifier 201, feedback resistor Rf, output node Vo that provides output Vo, p-type bias transistor MPbiasP, n-type cascode device MNcascode, n-type bias transistor MNbiasN, and photodiode 202. In some embodiments, amplifier 201 is configured as a TIA. In some embodiments, the negative input terminal of amplifier 201 is coupled to transistors MPbiasP and MNcascode. Here, the photodiode 202 together with transistor MNbias can be considered as a current source. In some embodiments, photodiode 202 alone can be considered a current source. In some embodiments, transistor MNcascode is in series with transistor MNbiasN. In some embodiments, photodiode 202 is coupled in parallel to transistor MNbiasN. In some embodiments, the positive input terminal of amplifier 201 is coupled to a reference Vref (e.g., VDDA/2, where VDDA is the power supply voltage which is provided by any suitable power supply source). In various embodiments, transistor MNcascode provides for a common-gate stage with its output coupled to the negative terminal of amplifier 201 while its input is coupled to photodiode 202. In various embodiments, transistor MNcascode provides a function of voltage or current buffer and thus can isolate parasitic elements (e.g., parasitic capacitance) of photodiode 202 from amplifier 201.

In some embodiments, bias circuitry (not shown) is used to provide bias voltages Pbias, Nbias, and Vcascode to bias transistors MPbiasP, MNbiasN, and MNcascode for particular current and gain amplification. Any suitable bias circuitry (e.g., bandgap, resistor divider, etc.) can be used for providing the biases. The biases can be static (e.g., predetermined) or programmable (e.g., by hardware such as fuses and/or resistors, or by software such as an operating system).

The instability of the conventional TIA apparatus 200 can be addressed by adding a common-gate stage comprising transistor MNcascode, in accordance with some embodiments. In one such embodiment, the photodiode capacitance of photodiode 202 is effectively isolated from the TIA. In some embodiments, the current-noise from transistors MPbiasP and MNbiasN (iNoiseN and InoiseP, respectively) is gained by the feedback resistance Rf directly to the output of the TIA.

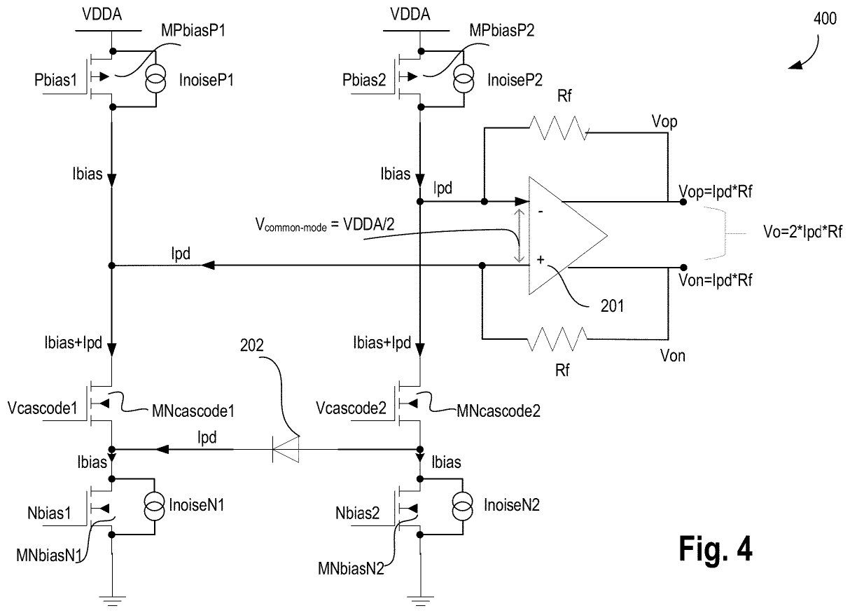

FIG. 4 illustrates apparatus 400 with fully-differential common-gate stage to effectively reduce photodiode capacitance, in accordance with some embodiments. The apparatus of FIG. 4 is a fully-differential version of apparatus of FIG. 3, in accordance with some embodiments. In some cases, the apparatus of FIG. 4 results in more noise sources that are gained by the feedback resistance Rf.

In some embodiments, apparatus 400 comprises p-type transistors MPbiasP1 and MPbiasP2, n-type transistors MNbiasN1, MNbiasN2, MNcascode1, and MNcascode2, amplifier 201, feedback resistors Rf, and photodiode 202. The fully-differential common-gate stage of apparatus 400 comprises two single common-gate stages, each for an input of amplifier 201, where each common-gate stage is similar to the circuitry comprising common-gate stage of apparatus 300. Here, the first common-mode gate stage comprises transistors MPbiasP1, MNcascode1, and MNbiasN1 coupled together in series, where transistor MBbiasP1 is biased by Pbias1, transistor MNcascode1 is biased by Vcascode1, and transistor MNbiasN1 is biased by Nbias1. Here, the noise source of transistor MPbiasP1 is illustrated with reference to InoiseP1, and the noise source of transistor MNbiasN1 is illustrated with reference to InoiseN1. The second common-mode gate stage comprises transistors MPbiasP2, MNcascode2, and MNbiasN2 coupled together in series, where transistor MPbiasP2 is biased by Pbias2, transistor MNcascode2 is biased by Vcascode2, and transistor MNbiasN2 is biased by Nbias2. Here, the noise source of transistor MPbiasP2 is illustrated with reference to InoiseP2, and noise source of MNbiasN2 is illustrated with reference to InoiseN2.

In the fully-differential common-gate stage of apparatus 400, the photodiode 202 is coupled to both common-mode gate stages. Two separate feedback resistors Rf are also provided for the fully-differential common-gate stage of apparatus 400, where each feedback resistor Rf couples a respective output to a respective input of amplifier 201. For example, output Vop is coupled to the negative terminal of amplifier 202, while output Von is coupled to the positive terminal of amplifier 201, where each output provides a voltage proportional to a product of diode current Ipd and resistance Rf. The resistive devices of various embodiments can be implemented with any known and suitable technology. For example, resistive devices may be specific resistors of a process node, or can be transistors configured to operate in a linear mode.

In some embodiments, bias circuitry (not shown) is used to provide bias voltages Pbias1, Nbias1, and Vcascode1 to bias transistors MPbiasP1, MNbiasN1, and MNcascode1, respectively, for particular current and gain amplification. In some embodiments, the same or different bias circuitry (not shown) is used to provide bias voltages Pbias2, Nbias2, and Vcascode2 to bias transistors MPbiasP2, MNbiasN2, and MNcascode2, respectively, for particular current and gain amplification. In some embodiments, the bias voltages Pbias1, Nbias1, and Vcascode1 can be substantially the same (or even identical) as bias voltages Pbias2, Nbias2, and Vcascode2, respectively. In some embodiments, bias voltages Pbias1, Nbias1, and Vcascode1 can be different than bias voltages Pbias2, Nbias2, and Vcascode2, respectively. Any suitable bias circuitry (e.g., bandgap, resistor divider, etc.) can be used for providing the biases. The biases can be static (e.g., predetermined) or programmable (e.g., by hardware such as fuses and/or resisters, or by software such as operating system).

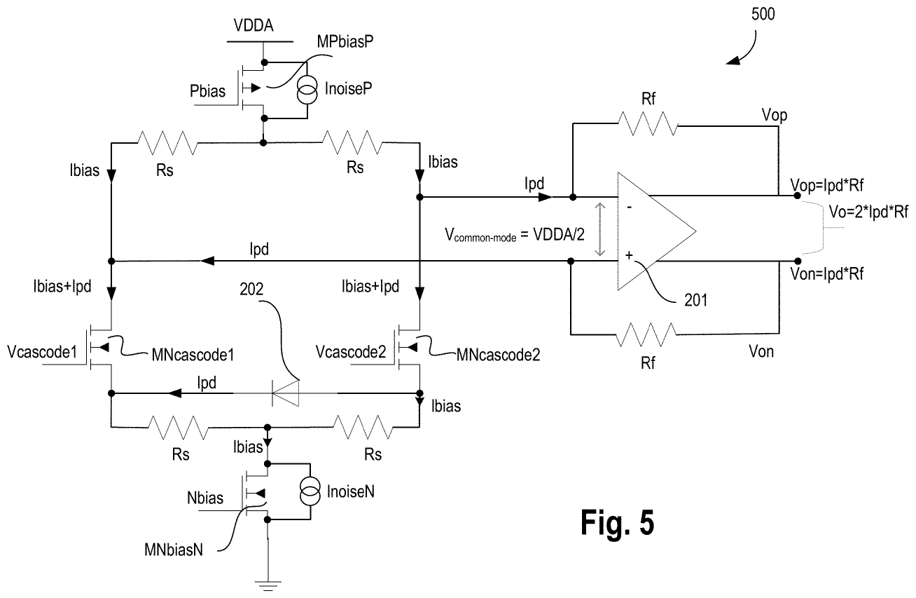

FIG. 5 illustrates apparatus 500 with current splitting resistors based fully-differential common-gate state to effectively reduce photodiode capacitance, in accordance with some embodiments. Apparatus 500 is similar to apparatus 400 but for the addition of splitting resistors Rs and common current sources MPbiasP and MNBiasN, that are biased by Pbias and Nbias respectively. The splitting resistors Rs couple the two common-gate stages (e.g., comprising MNcascode1 and MNcascode2) as shown. In some embodiments, to mitigate the problem of noise gain by the apparatus 400 of FIG. 4, current splitting resistors Rs are added that make the noise common mode. In some embodiments, the current splitting resistors Rs do not affect the current-gain of the TIA since to a first order this is Ipd*Rf. The current-noise from these cascode devices, MNcascode1 and MNcascode2, is now degenerated by current splitting resistors Rs. The apparatus of FIG. 5 results in improved accuracy of heart-rate measurement in optical heart rate monitors (e.g., fitness watches).

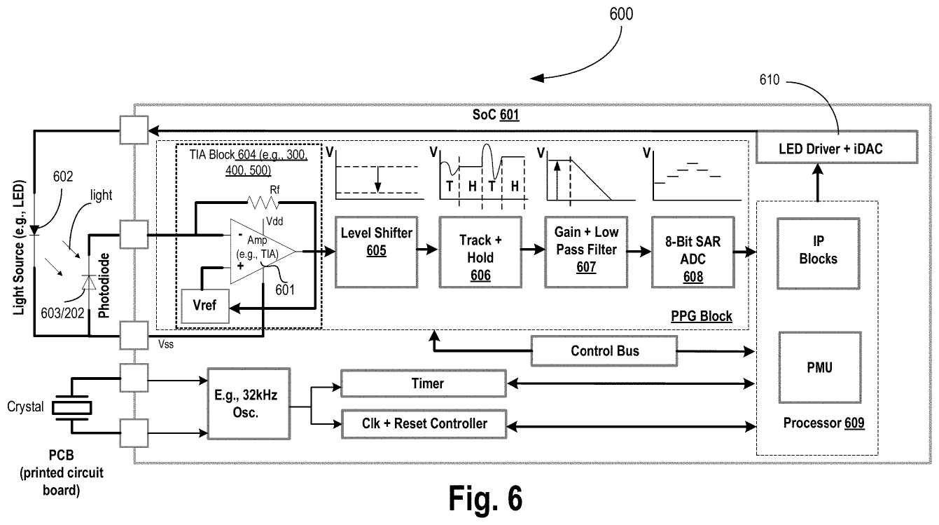

FIG. 6 illustrates a SoC (System-on-Chip) 600 with a PPG device with common-gate stage to effectively reduce photodiode capacitance, according to some embodiments of the disclosure.

In some embodiments, apparatus 600 is part of a wearable device (e.g., a smartwatch, heart rate monitor). In some embodiments, apparatus 600 comprises SoC 601, light source (e.g., LED) 602, current-source (e.g., photodiode) 603 (e.g., 202), Amplifier 604, Level Shifter 605, Track and Hold circuit 606, Amplifier (i.e., Gain stage) and Low Pass Filter (LPF) 607, Analog-to-Digital Converter (ADC) 608, Processor 609, LED Driver and current Digital-to-Analog Converter (iDAC) 610, Crystal for providing a periodic clock signal, Oscillator, Timer, Clock (Clk) and Reset Controller, and Control Bus as shown. Apparatus 600 may have fewer or more components than does listed here.

The term “light source” generally refers to a source that may provide visible light (e.g., visible to the human eye and having wavelengths in the range of 400 nm to 700 nm) or invisible light (e.g., invisible to the human eye and having wavelengths outside the range of 400 nm to 700 nm).

Various embodiments here are described with reference to the amplifier in Block 604 being a TIA. However, other implementations of the amplifier are also possible. Various embodiments here are described with reference to the light source being an LED. However, other implementations of the light source are also possible. Various embodiments here are described with reference to the current-source in being a photodiode. However, other implementations of the current-source are also possible.

In some embodiments, current (e.g., LED current) is driven by the light source (e.g., LED driver) in response to controls provided by Processor 609. For example, the controls provided by Intellectual Property (IP) block(s), of Processor 609, for the LED driver may set the Pulse Repetition Frequency (PRF), light intensity, duty cycle ratio, and other attributes of LED 602. Here the term “IP Block” refers to a reusable unit of logic, cell, or integrated circuit (commonly called a “chip”) layout design that is the intellectual property of one party. IP blocks may be licensed to another party or can be owned and used by a single party alone.

In some embodiments, the PRF of LED 602 is set low (e.g., several Hertz). In some embodiments, the duty cycle ratio is also set low (e.g., 100:1). For example, the off-time of LED 602 has a longer duration than the on-time of LED 602. In some embodiments, this control timing scheme of LED 602 allows conservation of power because LED 602 consumes hundreds of milli-Amperes (mA). In some embodiments, photodiode 603 (or 202) is an off-chip diode which receives the light reflected off the user's wrist. In some embodiments, photodiode 603/202 is integrated in SoC 601 such that it is able to receive light.

In some embodiments, the current generated by photodiode 603 is received by Amplifier Block 604. For example, the current corresponding to the pulsed light transmitted by LED 202 and reflected off from the organs or bones of the user's wrist is received by Amplifier Block 604. In some embodiments, Amplifier Block 604 includes common-gate stage to effectively reduce photodiode capacitance using schemes described with reference to FIGS. 3-5.

Referring back to FIG. 6, Amplifier Block 604 generates a voltage output which is level shifted down to a suitable common mode and held between pulses of LED 602 by Track and Hold Filter 606. Any suitable circuit can be used for implementing Track and Hold Filter 606. The output of Track and Hold Filter 606 are stripped-out AC waveforms (e.g., portions of the AC waveforms), according to some embodiments. In some embodiments, the output of Track and Hold Filter 606 are presented to an active second order low pass filter (e.g., Gain and Low Pass Filter 607). In some embodiments, Gain and Low Pass Filter 607 includes an amplifier to amplify the output of Track and Hold Filter 606 and to filter the high frequency AC component from the output. In some embodiments, Gain and Low Pass Filter 607 has enough gain to excite ADC 608. In some embodiments, ADC 608 is a Successive Approximation Register (SAR) based analog-to-digital converter (ADC).

SAR based ADC 608 is a type of ADC that converts a continuous analog waveform (e.g., filtered output of Gain and Low Pass Filter 607) into a discrete digital representation via a binary search through all possible quantization levels before finally converging upon a digital output for each conversion. In some embodiments, ADC 608 is designed such that poles are placed to limit aliasing. In some embodiments, ADC 608 is 8-bit SAR topology sampling at around 100 Hz and using around 20 dB of gain after trans-impedance of about 1.8 MegR. In other embodiments other types of ADCs may be used to digitize the filtered content from Gain and Low Pass Filter 607.

In some embodiments, ADC 608 is switched on to sample the signal presented by the chain (i.e., blocks 603, 604, 605, 606, and 607) when LED 602 is pulsed on. In some embodiments, the entire system can be shut down between LED on phases to conserve battery power. For example, when LED 602 is off, the detection mechanism having Amplifier Block 604 along with other components may be turned off to conserve power. In some embodiments, the DC information required to track the signal at the next on phase is held on integrated MOS capacitors. Here, leakage may not be a major concern with this system as merely small portions of the large DC levels held may leak away, and not the signal of interest itself.

In some embodiments, Processor 609 processes the output of ADC 608 to generate a result (e.g., heartbeat, pulse rate, blood pressure, etc.). In some embodiments, Processor 609 may include Power Management Unit (PMU) to manage the power consumption of various blocks of SoC 601. In some embodiments, Processor 609 includes a plurality of Intellectual Property (IP) Blocks such as caches, memory controller, register files, input-output circuits, execution units, etc. In some embodiments, Processor 609 controls various attributes of LED 602, such as the strength of light generated by LED 602, by controlling LED Driver and current DAC (iDAC) 610.

In some embodiments, an oscillator (osc.) such as a 32 kHz osc is provided to illustrate the low clock frequency uses of this circuitry (and therefore low power). In some embodiments, Timer/reset controller are generic features associated with a generated clock. In some embodiments, the control bus is intended to be a digital interface between Processor 609 and the PPG Block. In some embodiments, the Control Bus can be used to trim values, control lines, and/or enables, any form of logic level information that may be passed to and from the PPG Block.

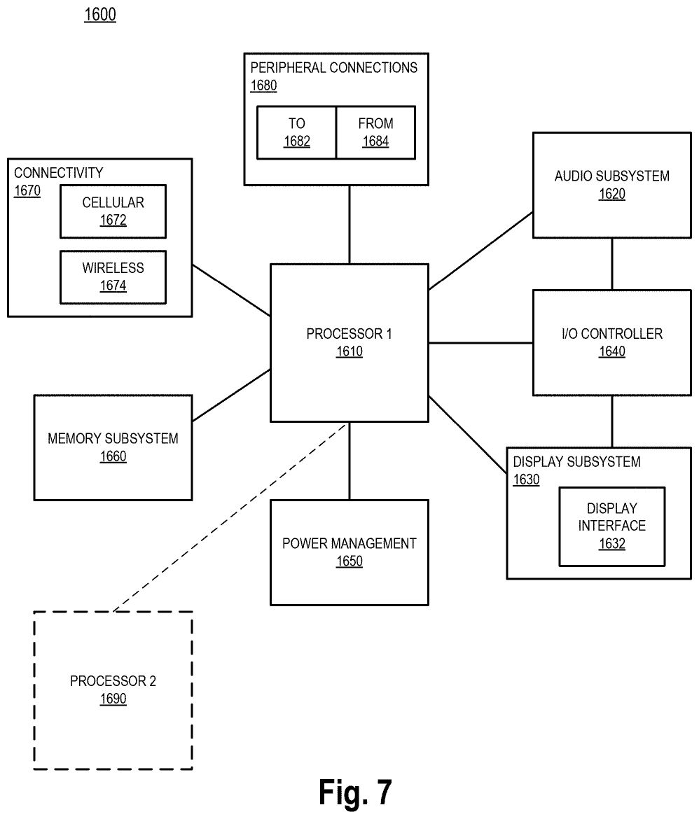

FIG. 7 illustrates a smart device or a computer system or a SoC (System-on-Chip) having a common-gate stage to effectively reduce photodiode capacitance, according to some embodiments of the disclosure. FIG. 7 illustrates a block diagram of an embodiment of a mobile device in which flat surface interface connectors could be used. In some embodiments, computing device 1600 represents a mobile computing device, such as a computing tablet, a mobile phone or smart-phone, a wireless-enabled e-reader, or other wireless mobile device. It will be understood that certain components are shown generally, and not all components of such a device are shown in computing device 1600.

In some embodiments, computing device 1600 includes first processor 1610 having a common-gate stage to effectively reduce photodiode capacitance, according to some embodiments discussed. Other blocks of the computing device 1600 may also include a common-gate stage to effectively reduce photodiode capacitance, according to some embodiments. The various embodiments of the present disclosure may also comprise a network interface within 1670 such as a wireless interface so that a system embodiment may be incorporated into a wireless device, for example, cell phone or personal digital assistant.

In some embodiments, processor 1610 (and/or processor 1690) can include one or more physical devices, such as microprocessors, application processors, microcontrollers, programmable logic devices, or other processing means. The processing operations performed by processor 1610 include the execution of an operating platform or operating system on which applications and/or device functions are executed. The processing operations include operations related to I/O (input/output) with a human user or with other devices, operations related to power management, and/or operations related to connecting the computing device 1600 to another device. The processing operations may also include operations related to audio I/O and/or display I/O.

In some embodiments, computing device 1600 includes audio subsystem 1620, which represents hardware (e.g., audio hardware and audio circuits) and software (e.g., drivers, codecs) components associated with providing audio functions to the computing device. Audio functions can include speaker and/or headphone output, as well as microphone input. Devices for such functions can be integrated into computing device 1600, or connected to the computing device 1600. In one embodiment, a user interacts with the computing device 1600 by providing audio commands that are received and processed by processor 1610.

In some embodiments, computing device 1600 comprises display subsystem 1630. Display subsystem 1630 represents hardware (e.g., display devices) and software (e.g., drivers) components that provide a visual and/or tactile display for a user to interact with the computing device 1600. Display subsystem 1630 includes display interface 1632, which includes the particular screen or hardware device used to provide a display to a user. In one embodiment, display interface 1632 includes logic separate from processor 1610 to perform at least some processing related to the display. In one embodiment, display subsystem 1630 includes a touch screen (or touch pad) device that provides both output and input to a user.

In some embodiments, computing device 1600 comprises I/O controller 1640. I/O controller 1640 represents hardware devices and software components related to interaction with a user. I/O controller 1640 is operable to manage hardware that is part of audio subsystem 1620 and/or display subsystem 1630. Additionally, I/O controller 1640 illustrates a connection point for additional devices that connect to computing device 1600 through which a user might interact with the system. For example, devices that can be attached to the computing device 1600 might include microphone devices, speaker or stereo systems, video systems or other display devices, keyboard or keypad devices, or other I/O devices for use with specific applications such as card readers or other devices.

As mentioned above, I/O controller 1640 can interact with audio subsystem 1620 and/or display subsystem 1630. For example, input through a microphone or other audio device can provide input or commands for one or more applications or functions of the computing device 1600. Additionally, audio output can be provided instead of, or in addition to, display output. In another example, if display subsystem 1630 includes a touch screen, the display device also acts as an input device, which can be at least partially managed by I/O controller 1640. There can also be additional buttons or switches on the computing device 1600 to provide I/O functions managed by I/O controller 1640.

In some embodiments, I/O controller 1640 manages devices such as accelerometers, cameras, light sensors or other environmental sensors, or other hardware that can be included in the computing device 1600. The input can be part of direct user interaction, as well as providing environmental input to the system to influence its operations (such as filtering for noise, adjusting displays for brightness detection, applying a flash for a camera, or other features).

In some embodiments, computing device 1600 includes power management 1650 that manages battery power usage, charging of the battery, and features related to power saving operation. Memory subsystem 1660 includes memory devices for storing information in computing device 1600. Memory can include nonvolatile (state does not change if power to the memory device is interrupted) and/or volatile (state is indeterminate if power to the memory device is interrupted) memory devices. Memory subsystem 1660 can store application data, user data, music, photos, documents, or other data, as well as system data (whether long-term or temporary) related to the execution of the applications and functions of the computing device 1600.

Elements of embodiments are also provided as a machine-readable medium (e.g., memory 1660) for storing the computer-executable instructions (e.g., instructions to implement any other processes discussed herein). The machine-readable medium (e.g., memory 1660) may include, but is not limited to, flash memory, optical disks, CD-ROMs, DVD ROMs, RAMs, EPROMs, EEPROMs, magnetic or optical cards, phase change memory (PCM), or other types of machine-readable media suitable for storing electronic or computer-executable instructions. For example, embodiments of the disclosure may be downloaded as a computer program (e.g., BIOS) which may be transferred from a remote computer (e.g., a server) to a requesting computer (e.g., a client) by way of data signals via a communication link (e.g., a modem or network connection).

In some embodiments, computing device 1600 comprises connectivity 1670. Connectivity 1670 includes hardware devices (e.g., wireless and/or wired connectors and communication hardware) and software components (e.g., drivers, protocol stacks) to enable the computing device 1600 to communicate with external devices. The computing device 1600 could be separate devices, such as other computing devices, wireless access points or base stations, as well as peripherals such as headsets, printers, or other devices.

Connectivity 1670 can include multiple different types of connectivity. To generalize, the computing device 1600 is illustrated with cellular connectivity 1672 and wireless connectivity 1674. Cellular connectivity 1672 refers generally to cellular network connectivity provided by wireless carriers, such as provided via GSM (global system for mobile communications) or variations or derivatives, CDMA (code division multiple access) or variations or derivatives, TDM (time division multiplexing) or variations or derivatives, or other cellular service standards. Wireless connectivity (or wireless interface) 1674 refers to wireless connectivity that is not cellular, and can include personal area networks (such as Bluetooth, Near Field, etc.), local area networks (such as Wi-Fi), and/or wide area networks (such as WiMax), or other wireless communication.

In some embodiments, computing device 1600 comprises peripheral connections 1680. Peripheral connections 1680 include hardware interfaces and connectors, as well as software components (e.g., drivers, protocol stacks) to make peripheral connections. It will be understood that the computing device 1600 could both be a peripheral device (“to” 1682) to other computing devices, as well as have peripheral devices (“from” 1684) connected to it. The computing device 1600 commonly has a “docking” connector to connect to other computing devices for purposes such as managing (e.g., downloading and/or uploading, changing, synchronizing) content on computing device 1600. Additionally, a docking connector can allow computing device 1600 to connect to certain peripherals that allow the computing device 1600 to control content output, for example, to audiovisual or other systems.

In addition to a proprietary docking connector or other proprietary connection hardware, the computing device 1600 can make peripheral connections 1680 via common or standards-based connectors. Common types can include a Universal Serial Bus (USB) connector (which can include any of a number of different hardware interfaces), DisplayPort including MiniDisplayPort (MDP), High Definition Multimedia Interface (HDMI), Firewire, or other types.

Reference in the specification to “an embodiment,” “one embodiment,” “some embodiments,” or “other embodiments” means that a particular feature, structure, or characteristic described in connection with the embodiments is included in at least some embodiments, but not necessarily all embodiments. The various appearances of “an embodiment,” “one embodiment,” or “some embodiments” are not necessarily all referring to the same embodiments. If the specification states a component, feature, structure, or characteristic “may,” “might,” or “could” be included, that particular component, feature, structure, or characteristic is not required to be included. If the specification or claim refers to “a” or “an” element, that does not mean there is only one of the elements. If the specification or claims refer to “an additional” element, that does not preclude there being more than one of the additional element.

Furthermore, the particular features, structures, functions, or characteristics may be combined in any suitable manner in one or more embodiments. For example, a first embodiment may be combined with a second embodiment anywhere the particular features, structures, functions, or characteristics associated with the two embodiments are not mutually exclusive.

While the disclosure has been described in conjunction with specific embodiments thereof, many alternatives, modifications and variations of such embodiments will be apparent to those of ordinary skill in the art in light of the foregoing description. The embodiments of the disclosure are intended to embrace all such alternatives, modifications, and variations as to fall within the broad scope of the appended claims.

In addition, well known power/ground connections to integrated circuit (IC) chips and other components may or may not be shown within the presented figures, for simplicity of illustration and discussion, and so as not to obscure the disclosure. Further, arrangements may be shown in block diagram form in order to avoid obscuring the disclosure, and also in view of the fact that specifics with respect to implementation of such block diagram arrangements are highly dependent upon the platform within which the present disclosure is to be implemented (i.e., such specifics should be well within purview of one skilled in the art). Where specific details (e.g., circuits) are set forth in order to describe example embodiments of the disclosure, it should be apparent to one skilled in the art that the disclosure can be practiced without, or with variation of, these specific details. The description is thus to be regarded as illustrative instead of limiting.

The following examples pertain to further embodiments. Specifics in the examples may be used anywhere in one or more embodiments. All optional features of the apparatus described herein may also be implemented with respect to a method or process.

Example 1. An apparatus comprising: an amplifier; a first current source; a first pair of resistors coupled to the first current source and the amplifier; a pair of devices coupled to the first pair of resistors; a photo-diode coupled to the pair of devices; a second pair of resistors coupled to the pair of devices and the photo-diode; and a second current source coupled to the second pair of resistors.

Example 2. The apparatus of example 1, wherein the amplifier comprises a first input coupled to a first resistor of the first pair and to a first device of the pair of devices.

Example 3. The apparatus of example 2, wherein the amplifier comprises a second input coupled to a second resistor of the first pair and to a second device of the pair of devices.

Example 4. The apparatus of example 3, wherein the amplifier comprises a first output coupled to a third resistor, wherein the third resistor is coupled to the first input of the transimpedance amplifier.

Example 5. The apparatus of example 3, wherein the amplifier comprises a second output coupled to a fourth resistor, wherein the fourth resistor is coupled to the second input of the transimpedance amplifier.

Example 6. The apparatus of example 1, wherein the first current source comprises a p-type transistor.

Example 7. The apparatus of example 1, wherein the second current source comprises an n-type transistor.

Example 8. The apparatus of example 1, wherein the pair of devices comprises common-gate amplifiers.

Example 9. The apparatus of example 1, wherein the amplifier comprises a transimpedance amplifier.

Example 10. An apparatus comprising: an amplifier having an input and an output; a resistor coupled to the input and the output of the amplifier; a first current source coupled to the input of amplifier; a second current source; a cascode device coupled to the first and second current sources; and a photo-diode coupled to the cascode device and the second current source.

Example 11. The apparatus of example 10, wherein the amplifier comprises a transimpedance amplifier.

Example 12. The apparatus of example 10, wherein the cascode device comprises a common-gate amplifier.

Example 13. The apparatus of example 10, wherein the first current source comprises a p-type transistor, and wherein the second current source comprises an n-type transistor.

Example 14. A wearable device comprising: a first current source to detect a light from a media; a cascode device coupled in series with the current source; a second current source coupled in series with the cascode device; an amplifier coupled to the second current source and the cascode device, wherein the amplifier is to provide an output; and a processing intellectual property (IP) block to receive a filtered version of the output of the amplifier and to determine a condition of the media according to the output of the amplifier.

Example 15. The wearable device of example 14, comprises a wireless interface to allow the processing IP block to communicate with another device.

Example 16. The wearable device of example 14 comprises: a level shifter to level shift the output to a lower voltage level; and a track-and-hold circuit to track the level-shifted output voltage and then to hold it.

Example 17. The wearable device of example 16 comprises a gain stage with a low pass filter, wherein the gain stage is to amplify the output of the track-and-hold circuit and is to filter the amplified output.

Example 18. The wearable device of example 17 comprises an analog-to-digital converter to convert the filtered amplified output to a digital representation which is the filtered version of the output voltage provided to the processing IP block.

Example 19. The wearable device of example 14 comprises a light source driver, wherein the processing IP block is operable to adjust intensity of the light emitted by the light source by controlling the light source driver.

Example 20. The wearable device of example 14, wherein the media is part of a living body, and wherein the condition is a heartbeat.

Example 21. The wearable device of example 14, wherein the first current source comprises a photodiode coupled in parallel to an n-type device.

Example 22. A system comprising: a memory, a processor coupled to a memory, the processor including an apparatus according to any one of examples 1 to 9; a wireless interface to allow the processor to communicate with another device.

Example 23. A system comprising: a memory, a processor coupled to a memory, the processor including an apparatus according to any one of examples 10 to 13; a wireless interface to allow the processor to communicate with another device.

An abstract is provided that will allow the reader to ascertain the nature and gist of the technical disclosure. The abstract is submitted with the understanding that it will not be used to limit the scope or meaning of the claims. The following claims are hereby incorporated into the detailed description, with each claim standing on its own as a separate embodiment.

What is claimed is:

1-23. (canceled)

24. An apparatus comprising:
an amplifier;
a first current source;
a first pair of resistors coupled to the first current source and the amplifier;
a pair of devices coupled to the first pair of resistors;
a photo-diode coupled to the pair of devices;
a second pair of resistors coupled to the pair of devices and the photo-diode; and
a second current source coupled to the second pair of resistors.

25. The apparatus of claim 24, wherein the amplifier comprises a first input coupled to a first resistor of the first pair and to a first device of the pair of devices.

26. The apparatus of claim 25, wherein the amplifier comprises a second input coupled to a second resistor of the first pair and to a second device of the pair of devices.

27. The apparatus of claim 26, wherein the amplifier comprises a first output coupled to a third resistor, and wherein the third resistor is coupled to the first input of the amplifier.

28. The apparatus of claim 25, wherein the amplifier comprises a second output coupled to a fourth resistor, and wherein the fourth resistor is coupled to the second input of the amplifier.

29. The apparatus of claim 24, wherein the first current source comprises a p-type transistor.

30. The apparatus of claim 24, wherein the second current source comprises an n-type transistor.

31. The apparatus of claim 24, wherein the pair of devices comprises common-gate amplifiers.

32. The apparatus of claim 24, wherein the amplifier comprises a trans-impedance amplifier.

33. An apparatus comprising:
an amplifier having an input and an output;
a resistor coupled to the input and the output of the amplifier;
a first current source coupled to the input of amplifier;
a second current source;
a cascode device coupled to the first and second current sources; and
a photo-diode coupled to the cascode device and the second current source.

34. The apparatus of claim 33, wherein the amplifier comprises a trans-impedance amplifier.

35. The apparatus of claim 33, wherein the cascode device comprises a common-gate amplifier.

36. The apparatus of claim 33, wherein the first current source comprises a p-type transistor, and wherein the second current source comprises an n-type transistor.

37. A wearable device comprising:
a first current source to detect a light from a media;
a cascode device coupled in series with the current source;
a second current source coupled in series with the cascode device;
an amplifier coupled to the second current source and the cascode device, wherein the amplifier is to provide an output; and
an intellectual property (IP) block to receive a filtered version of the output of the amplifier and to determine a condition of the media according to the output of the amplifier.

38. The wearable device of claim 37, comprises a wireless interface to allow the IP block to communicate with another device.

39. The wearable device of claim 37 comprises:
a level shifter to level shift the output to a lower voltage level; and
a track-and-hold circuit to track the level-shifted output voltage and then to hold it.

40. The wearable device of claim 39 comprises a gain stage with a low pass filter, wherein the gain stage is to amplify the output of the track-and-hold circuit and is to filter the amplified output.

41. The wearable device of claim 40 comprises an analog-to-digital converter to convert the filtered amplified output to a digital representation, which is the filtered version of the output voltage provided to the IP block.

42. The wearable device of claim 37 comprises a light source driver, wherein the IP block is operable to adjust intensity of the light emitted by the light source by controlling the light source driver.

43. The wearable device of claim 37, wherein:
the media is part of a living body;
the condition is a heartbeat; and
the first current source comprises a photodiode coupled in parallel to an n-type device.
